(19) United States
(12) Patent Application Publication
(10) Pub. No.: US 2003/0086959 A1
(43) Pub. Date: May 8, 2003

(54) Animal feed containing simple polysaccharides

(76) Inventors: Sangki Rhee, Seoul (KR); Kibang Song, Taejeon (KR); Chulho Kim, Taejeon (KR); Byoungdae Yoon, Taejeon (KR)

Correspondence Address: PENNIE AND EDMONDS, 1155 AVENUE OF THE AMERICAS, NEW YORK, NY, 100362711

(21) Appl. No.: 10/169,613

(22) PCT Filed: Dec. 29, 2000
(86) PCT No.: PCT/KR00/01556

(30) Foreign Application Priority Data
Jan. 7, 2000 (KR) 2000/000646

(51) Int. Cl. A61K 47/00

(52) U.S. Cl. 424/439

(57) ABSTRACT

The present invention relates to an animal feed containing the 0.04-2 (w/w) % simple polysaccharide on the basis of solid powder. The animal feed according to this invention is effective in improving the condition of evacuation, preventing the diarrhea and promoting the growth of animals.

### TECHNICAL FIELD

The present invention relates to animal feed containing simple polysaccharide. In detailed description, the present invention relates to animal feed containing a proper amount of simple polysaccharide, which can improve the condition of evacuation, prevent diarrhea and further promote animal's growth

### BACKGROUND ART

Stress on animals, caused by changes in weaning food, animal feed or breeding environment, is often the reason of their diarrhea or fluid stool and their retarded growth. Particularly, young domestic animals in the weaning stage are sensitive to change of the environment, and prone to stress and diarrhea therefrom. This condition influences a total period of their growth, resulting in extension of the growth period and increase of the breeding cost.

In order to prevent and treat diarrhea or fluid stool causing an economic loss of livestock farms, several antibiotics, microorganism (Lactobacillus or Bifidobacterium), or oligosaccharide, disclosed in prior art (KR Open Publication No. 90-17493, KR Publication No. 95-4426, JP Publication No. 79-15829, KR Publication No. 94-957, KR Patent No. 171,246, and KR Patent No. 198,727), have been administered.

However, in case of a long-term use of these antibiotics, its tolerance to antibiotics may be developed, beneficial intestinal cells in the animal also may be destroyed, and some control of the shipping time of the animal is needed because of the antibiotics' remains in the animal.

Also, in case of administration of beneficial microorganism, enough amount of microorganism cannot reach to the large intestine due to simple death of the microorganism by gastric acid, and it is difficult for the foreign microorganism to survive in animal's intestine, so expected effect of using microorganism cannot be obtained.

The method of using lactose-containing powder prepared from milk whey (JP Publication No. 79-15829) or the method of using branched oligosacchride (KR Publication No. 95-4426) includes complicated processes and is not adequately effective. For examples, in the market, the product called Bio-Mos (Altech Corp.), which contains mannose oligosaccharide as a major component, is too expensive to be used in bulk in farms.

Considering the aforementioned problems of prior art, our studies have led to this invention with the result that if polysacchride, which is larger than oligosaccharide, be added, the animal feed can improve the condition of evacuation, prevent diarrhea and further promote animal's growth

It is an object of this invention to provide an animal feed improving the condition of evacuation, preventing the diarrhea and promoting the growth of animals.

### DISCLOSURE OF THE INVENTION

To achieve said purpose, this invention provides an animal feed containing the 0.04-2 (w/w) % simple polysaccharide on the basis of solid powder.

According to the invention, the examples of the simple polysaccharide are levan, dextran, alteman, mutan or inulin, and one of these or more as a mixture can be used in this invention.

Also, according to this invention, the aforementioned polysaccharide may be purchased as a refined form, or derived from sucrose using biological techniques. There is no specific limitation in the biological techniques, and the examples of the biological techniques are fermentation using microorganism or enzyme reaction method using enzymes from the fermentation broth. Batch, continuous or immobilized type can be used. The polysaccharide solution obtained by these methods may contain glucose, fructose, sucrose, or other oligosaccharide, can be purified using ion-exchange method, gel-filtration method, or membrane isolation method to obtain polysaccharide with high purity.

As the practical aspect, levan in this invention is a polysaccharide consisting of β-2,6 bonds between fructose molecules with 5-15% of β-1,2 bond content in branch chains, and may be prepared from sugar using levansucrase from microorganism such as Zymomonas mobilis, Bacillus sp., Rahnella aquatilis, and Gluconobacter sp., or plants Further, levan may be produced using recombinant levansucrase, whose coding gene has been purified, and transferred in microorganism such as E. coli, yeast or Bacillus subtilis, and which has been overexpressed

Sugar (sucrose) for production of polysaccharide in this invention is selected from th group consisting of refined sugar, yellow sugar, and raw sugar. Polysaccharide obtained by aforementioned methods may be in a conventional syrup type, and further by some proper treatments in a powder or granule type

According to this invention, the animal feed contains characteristically 0.04-2 (w/w) % of the simple polysaccharide on the basis of solid powder and probably some components of conventional animal feed. Also, according to this invention, the animal feed may comprise transgenic plants, which have been transformed with the gene encoding the polysaccharide-synthesizing enzyme and contain the polysaccharide. According to the conventional manufacturing processes of animal feed, the polysaccharide in this invention may be added/mixed to/with raw materials of the feed to a product in a proper type such as powder, mesh, or pellet; or the polysaccharide may be directly added/mixed to/with a prepared feed product, resulting in an animal feed

According to the present invention, the animal feed is suitable but not limited to livestock or domestic fowls such as oxen, pigs, horses, goats, fowls, ducks, dogs, cats, monkeys, or mice; probably to zoo animals such as lions, tigers, monkeys, etc; and preferably to mammals in lactation or poultry in insemination.

According to the present invention, the animal feed has effect to improve the condition of evacuation, prevent the diarrhea, and promote the growth of animals by affecting various factors relating to growth, for example, increasing beneficial microbes such as Bifidobacteria. Also, the animal feed has effect to increase infant animal's resistance to diseases, strengthen its immune system, suppress harmful bacteria and intestinal toxin, and prevent diseases.

### BEST MODES FOR CARRYING OUT THE INVENTION

The present invention is illustrated but by no means limited by the following examples.

Preparation 1

Recombinant E. coli BL21(DE3)/pEL12 (KCTC 8661P; KR Patent No 145946), which can overexpress levansucrase, was inoculated in M9-ZB medium (in 1 L, trypton 10 g, NaCl 5 g, NH4Cl 1 g, KH₂PO₄ 3 g, Na₂HPO₄6 g, glucose 4 g, 1 ml of 1M MgSO₄, and cultivated with shaking at 37° C. When OD (600 nm) of the culture medium reached to 0 7, expression of levanscurase was induced with 1 mM IPTG (SIGMA, USA). 3 hours after that, the cultured cells were isolated from culture medium with centrifigation, disrupted with sonication, centrifuged, and then the supernatant was used as the enzyme solution.

Preparation 2

2000 units of levansucrase solution obtained in preparation 1 was added to 20 L of 25% sugar solution, and then the mixture had been incubated at 4° C. for 7 days. Levan concentration produced in the reaction mixture was 43%, the saccharide composition was as following table 1

TABLE 1

| Composition | Content (%) |
|---|---|
| Levan | 43 |
| Glucose | 48 |
| Fructose | 6 |
| Unreacted Sugar | 3 |

Preparation 3

1000 units of levansucrase solution obtained in preparation 1 was added to 10 L of 25% sugar solution, and then the mixture had been incubated at 4° C. for 7 days. After the reaction, 10 L of the reaction mixture was added with 30 L of ethanol to precipitate, and then the precipitate was dried, resulting in 970 g of dry powder The composition of the dry powder is shown in following table 2.

TABLE 2

| Composition | Content (%) |
|---|---|
| Levan | 96 |
| Glucose | 2 |
| Fructose | 1 |
| Unreacted Sugar | 1 |

### EXAMPLE 1

In this experiment, 90-day-old pig, which had taken an assorted feed after ablactation, was used, and the feed was displaced by “humid-fermentated feed of food wastes”. Number 1 to 21 group was set and each number group comprised 14 members of pigs, that is, total 294 pigs were used for the experiment

In larger aspect, the pigs were classified into control groups (No. 1-3) and test groups (No. 4-21). The control groups had been supplied only with “humid-fermentated feed of food wastes”, and the test groups with the test feed, which contained 7 kg of “humid-fermentated feed of food wastes” and 20 ml (25% solid powder) of the reaction mixture prepared in the preparation 2 No. 4-10 pigs had ingested the test feed for 10 days; No. 11-17 pigs for 20 days; and No. 18-21 pigs for 110 days. These three groups were respectively identified as the experimental group 1, 2, and 3. All groups could take water freely, and the experiment had been carried out for 110 days after the test breeding, or 200 days after birth The weight change of the control and experimental groups (the weight before the test and after the test) and the change of the feces condition (before the test, 2 days, 7 days, and 15 days after the test) are shown in following table 3.

TABLE 3

| | | | Experimental group | | |
|---|---|---|---|---|---|
| | | | Group 1 | Group 2 | Group 3 |
| | | | (treatment | (treatment | (treatment |
| | | | for 10 | for 20 | for 110 |
| Classification | | Control | days) | days) | days) |
| Weight | Before the test | 35.5 ± 1 | | | |
| (kg) | At the end of | 114 ± 2 | 117.4 ± 2 | 117.6 ± 2 | 118.1 ± 2 |
| | the test | | | | |

| Condition | Before the test | 4 | |
|---|---|---|---|
| of the | After 2 days | 4 | 1 |
| feces * | After 7 days | 4 | 1 |
| | After 15 days. | 4 | 1 |

* Condition of the feces - 1 Hard, 2 Moderate, 3 diarrheal

As known from above table 3, the experimental groups, which had ingested the mixture containing the saccharide prepared according to the present invention, had their feces condition obviously improved in short administration period, and the weight increase of 2.9-3.6%, compared to the control group

### EXAMPLE 2

In this experiment, 7-day old pigs were used and the feed after ablactation had been displaced by “cramble feed (DODRAM FEED MILL CO., LTD.)”. Number 1 to 10 group was set and each number group comprised 5 members of pigs, that is, total 50 pigs were used for the experiment.

The pigs were classified into control groups (No. 1-2) and test groups (No. 3-10) The control groups had been supplied only with “cramble feed”, and the test groups with the test feed containing 2 g (0.04%; No. 3-4), 4 g (0.08%; No 5-6), 6 g (0.12%; No 7-8), and 10 g (0.2%; No. 9-10) of the dry powder prepared in preparation 2. These four groups were respectively identified as the experimental group 1, 2, 3, and 4. All group could take water freely, and 1 week after the experiment, the number of pigs having diarrhea was checked, as shown in table 4.

TABLE 4

| | Number of heads | Number of heads |
|---|---|---|
| | (Test) | (having diarrhea) |
| Control | 10 | 8 |
| Experimental group 1 | 10 | 2 |
| (0.04% powder) | | |
| Experimental group 2 | 10 | 0 |
| (0.08% powder) | | |
| Experimental group 3 | 10 | 0 |
| (0.12% powder) | | |
| Experimental group 4 | 10 | 0 |
| (0.2% powder) | | |

As known from above table 4, the experimental groups, which had ingested the mixture containing the saccharide prepared according to the present invention, had their feces condition obviously improved in short administration period, and the experimental group 2-4, that is, the test groups which had ingested the assorted feed with >0.08% of dry saccharide powder included no pig having diarrhea.

### INDUSTRIAL APPLICABILITY

The animal feed, according to the present invention, has excellent effects to improve the condition of evacuation, prevent the diarrhea, and promote the growth of animals, by affecting various factors relating to growth, for example, increasing beneficial microbes such as Bifidobacteria. Also, the animal feed has effects to increase infant animals resistance to diseases, strengthen its immune system, suppress harmful bacteria and intestinal toxin, and prevent diseases

Additionally, by this inventive animal feed, the feces of livestock may be improved to have moderate hardness, and therefore the disposal and cleaning of animal's feces will be easier. As a result, better sanitation of livestock and reduction of the malodor of pet's feces can be expected.

1. An animal feed containing 0.04-2 (w/w) % simple polysaccharide on the basis of solid powder

2. The animal feed according to claim 1, wherein the polysaccharide is contained by the transgenic plant which have been transformed with the gene encoding the simple-polysaccharide synthesis enzyme.

3. The animal feed according to claim 1 and claim 2, wherein the polysaccharide is levan, dextran, alternan, mutan, inulin, or a mixture thereof, or a mixture thereof with glucose, fructose, or sugar.

4. The animal feed according to claim 3, wherein levan is induced from sugar using levansucrase.
